(19) United States
(12) Patent Application Publication
(10) Pub. No.: US 2021/0401500 A1
(43) Pub. Date: Dec. 30, 2021

(54) EFFICIENT AUTOMATIC FINDING OF MINIMAL EAR-NOSE-THROAT (ENT) PATH FOR PROBE

(71) Applicant: BIOSENSE WEBSTER (ISRAEL) LTD., Yokneam (IL)

(72) Inventors: Yair Palti, Herzelia (IL); Vadim Gliner, Haifa (IL); Yoav Pinsky, Bet Keshet (IL)

(21) Appl. No.: 16/914,696

(22) Filed: Jun. 29, 2020

(51) Int. Cl. A61B 34/10; A61B 34/20; A61B 90/00

(52) U.S. Cl. CPC A61B 34/10; A61B 34/20; A61B 2090/374; A61B 2034/107; A61B 2090/3762; A61B 90/37

(57) ABSTRACT

A method includes receiving a medical imaging scan of at least a part of a body of a patient. Voxels of the scan are identified, that correspond to regions in the body that are traversable by a probe inserted therein. The scan is displayed on a screen and selected termination and start points for the probe are marked thereon. Using a processor, a backward path is found from the termination point to the start point comprising a connected set of the identified voxels. The backward path is visualized on the screen in association with the scan.

### FIELD OF THE INVENTION

This invention relates generally to surgery, and specifically to pre-planning of invasive nasal sinus surgery.

### BACKGROUND OF THE INVENTION

Various methods of planning surgical procedures were previously proposed in the patent literature. For example, U.S. Pat. No. 8,160,676 describes a method for planning a surgical procedure that can include a path or trajectory to reach a selected target.

As another example, U.S. Patent Application Publication 2008/0183073 describes methods to assist in planning routes through hollow, branching organs in patients to optimize subsequent endoscopic procedures.

U.S. Pat. No. 8,116,847 describes a method for calculating an optimum surgical trajectory or path for displacing a surgical instrument through the interior of the body of a patient.

U.S. Pat. No. 10,188,465 describes a method, consisting of receiving a computerized tomography scan of at least a part of a body of a patient, and identifying voxels of the scan that correspond to regions in the body that are traversable by a probe inserted therein. The method also includes displaying the scan on a screen and marking thereon selected start and termination points for the probe. A processor finds a path from the start point to the termination point consisting of a connected set of the identified voxels. The processor also uses the scan to generate a representation of an external surface of the body and displays the representation on the screen. The processor then renders an area of the external surface surrounding the path locally transparent in the displayed representation, so as to make visible on the screen an internal structure of the body in a vicinity of the path.

### SUMMARY OF THE INVENTION

An embodiment of the present invention that is described hereinafter provides a method including receiving a medical imaging scan of at least a part of a body of a patient. Voxels of the scan are identified, that correspond to regions in the body that are traversable by a probe inserted therein. The scan is displayed on a screen and selected termination and start points for the probe are marked thereon. Using a processor, a backward path is found from the termination point to the start point comprising a connected set of the identified voxels. The backward path is visualized on the screen in association with the scan.

In some embodiments, visualizing the backward path includes using the scan to generate a representation of an external surface of the body and displaying the representation on the screen. An area of the external surface surrounding the path is rendered locally transparent in the displayed representation, so as to make visible on the screen an internal structure of the body in a vicinity of the backward path.

In some embodiments, identifying the voxels of the scan includes selecting mucous as a traversable species.

In an embodiment, identifying the voxels of the scan includes identifying soft tissue as a traversable species.

In some embodiments, the medical imaging scan is a computerized tomography (CT) scan, and wherein identifying the voxels of the scan includes defining a range of Hounsfield units for voxels. In other embodiments, the medical imaging scan is a magnetic resonance (MR) scan, and identifying the voxels of the scan includes defining a range of MR image intensities for voxels.

In an embodiment, finding the backward path includes ensuring that no portion of the path includes a radius of curvature smaller than a range of possible radii of curvature of the probe. In another embodiment, finding the backward path includes ensuring that a path diameter is always larger than a diameter of the probe. In yet another embodiment, finding the backward path includes finding a shortest path from the termination point to the start point.

In some embodiments, finding the shortest backward path includes using Dijkstra's algorithm or an extension thereof.

In some embodiments, finding the backward path includes ensuring that the probe is not required to traverse a portion of the path having a path radius curvature smaller than a probe radius of curvature achievable at the portion.

There is additionally provided, in accordance with another embodiment of the present invention, an apparatus, including a screen and a processor. The screen is configured to display a medical imaging scan of at least a part of a body of a patient. The processor is configured to (i) receive the scan, (ii) identify voxels of the scan that correspond to regions in the body that are traversable by a probe inserted therein, (iii) mark on the screen selected termination and start points for the probe, (iv) find a backward path from the termination point to the start point comprising a connected set of the identified voxels, and (v) visualize the backward path on the screen in association with the scan.

The present disclosure will be more fully understood from the following detailed description of the embodiments thereof, taken together with the drawings, in which:

### BRIEF DESCRIPTION OF THE DRAWINGS

FIG. 1 is a schematic, pictorial illustration of a nasal sinus surgery system, according to an embodiment of the present invention;

FIG. 2 is a flow chart showing steps of a surgery pre-planning method carried out for the surgery system of FIG. 1, according to an embodiment of the present invention; and

FIGS. 3-8 are diagrams illustrating the steps of the method of FIG. 2, according to an embodiment of the present invention.

### DETAILED DESCRIPTION OF EMBODIMENTS

### Overview

The paranasal sinuses comprise four separate pairs of three-dimensional (3D) air-filled spaces which are in proximity to the nasal cavity. Invasive surgery of a selected region of the sinuses may be considered necessary, for example, in the case of severe sinusitis, when a probe, such as a catheter, is used to reach the region. In a manual pre-planning process, a computerized tomography (CT) scan of a selected region of one of the sinuses and its environs is taken prior to performing such invasive surgery. A physician analyzes the scan in order to select the best path, typically the shortest path, to be taken by the probe from a nostril to the selected region.

The selection of the best path, however, is not a trivial task. The sinuses are 3D spaces, and, especially if there is any sort of blockage between a nostril and the selected region, the best path may comprise a relatively complicated route. In addition, while the CT scan can be used to generate 3D images, the analysis of such three-dimensional images is both difficult and time-consuming.

Embodiments of the present invention that are described hereinafter provide a method for finding a preferred path for inserting a guidewire or catheter into the sinuses. The embodiments described herein refer mainly to finding the shortest path from a start point to a target point. The disclosed techniques, however, can be used in a similar manner to find paths that satisfy other requirements or constraints.

In the disclosed embodiments, rather than finding the preferred forward path from the start point to the target point, embodiments of the present invention find a preferred backward path (e.g., the shortest backward path) from the target point to the start point, i.e., in the reverse direction. By searching in the reverse direction, in many cases the preferred path can be found in significantly less time than using forward searching.

For example, the time to find a path from the nostril to the frontal sinus using the disclosed method was found to be up to nine times faster than using a forward-direction search, and from the nostril to the sphenoidal sinus three times faster. These reductions in search times taken in the reverse direction are due to the relatively small volumes of the target locations, with fewer possible initial partial backward paths to consider, whereas calculating a path in the forward direction must consider many more partial forward paths because of the large volume around the start point (the mouth).

The disclosed pre-planning technique typically starts with uploading a medical imaging scan of the region of the patient where the procedure is to be performed. In one embodiment, a CT scan of the procedure region is received, and voxels of the scan corresponding to regions of the body of the patient that are traversable by a probe to be inserted into the patient are identified. The identification is typically done by defining a range of Hounsfield units for the voxels.

The physician displays the scan on a screen, and marks termination and start points for the probe on the scan. Then a processor uses an algorithm, such as Dijkstra's algorithm, to find a backward path, typically the shortest backward path, from the termination point to the start point that has a connected set of the identified voxels.

The processor also generates a representation of an external surface of the body which is displayed on the screen. The processor then renders an area of the external surface surrounding the path locally transparent in the displayed representation, so as to make an internal structure of the body in a vicinity of the path visible on the screen.

The disclosed embodiments provide a physician who can afford only simple planning tools (e.g., tools having low computational capabilities) a cost-effective pre-planning surgery procedure to automatically select the best path to be taken by a catheter, and then displaying the selected path on a medical image of the patient.

### System Description

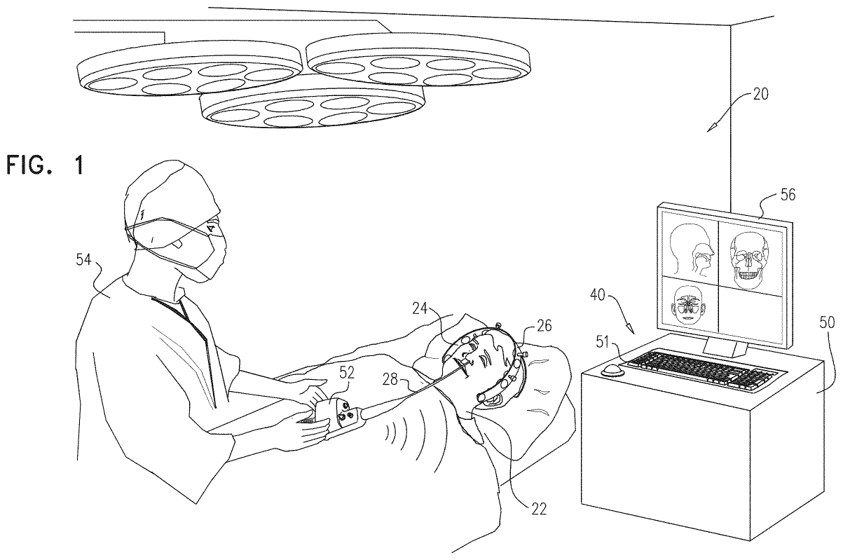

FIG. 1 is a schematic, pictorial illustration of a nasal sinus surgery system 20, according to an embodiment of the present invention. System 20 is typically used during an invasive procedure on a nasal sinus of a patient 22, and the system includes a surgery pre-planning component, described in more detail below.

For the actual procedure, a set of magnetic field generators 24 may be fixed to the head of the patient, for example by incorporating the generators into a frame 26 which is clamped to the patient's head. The field generators enable tracking of the position of a probe 28 that is inserted into the nasal sinus of the patient. A system using magnetic field generators, such as generators 24, for tracking a probe inserted into an organ of a patient is described in US Patent Application Publication 2016/0007842, which is incorporated herein by reference. In addition, the Carto® system produced by Biosense Webster, Irvine, Calif., uses a tracking system similar to that described herein for finding the location and orientation of a coil in a region irradiated by magnetic fields.

Elements of system 20, including generators 24, may be controlled by a system processor 40, comprising a processing unit communicating with one or more memories. Processor 40 may be mounted in a console 50, which comprises operating controls 51 that typically include a keypad and/or a pointing device such as a mouse or trackball. Console 50 also connects to other elements of system 20, such as a proximal end 52 of probe 28. A physician 54 uses the operating controls to interact with the processor while performing the procedure, and the processor may present results produced by system 20 on a screen 56.

Processor 40 uses software stored in a memory of the processor to operate system 20. The software may be downloaded to processor 40 in electronic form, over a network, for example, or it may, alternatively or additionally, be provided and/or stored on non-transitory tangible media, such as magnetic, optical, or electronic memory.

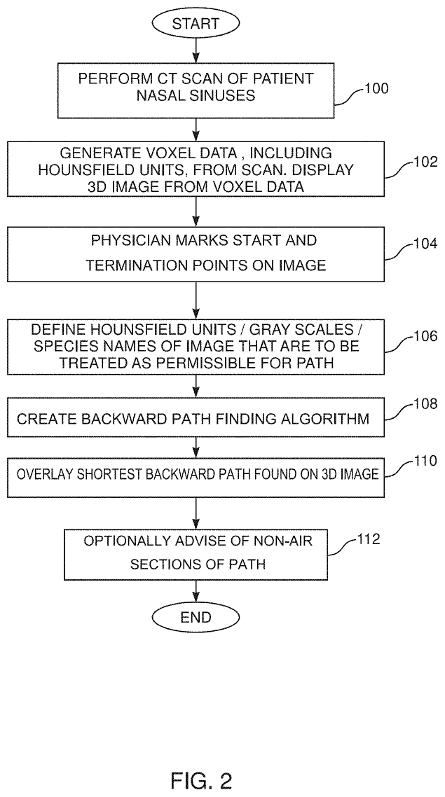

In particular, processor 40 runs a dedicated algorithm as disclosed herein, including in FIG. 2, that enables processor 40 to perform the disclosed steps, as further described below.

### Efficient Automatic Finding of Minimal Ear-Nose-Throat Ent Path for Probe

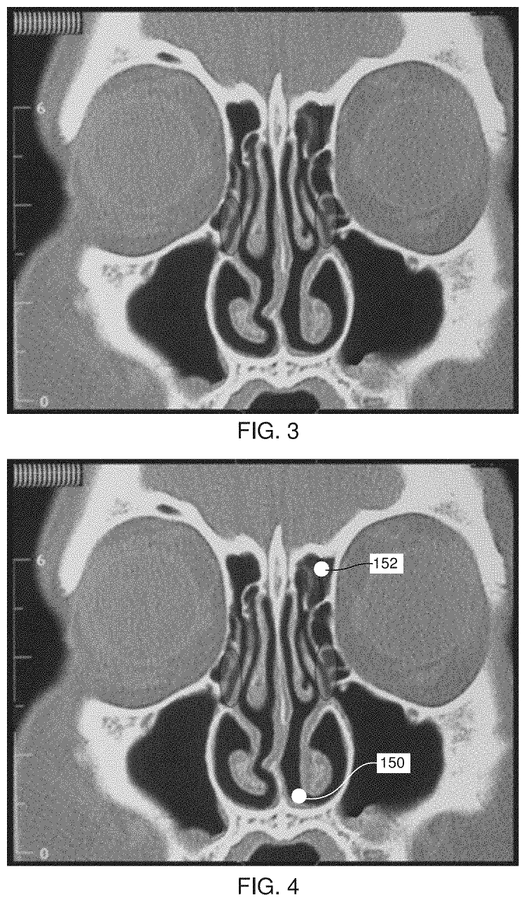

FIG. 2 is a flow chart showing steps of a surgery pre-planning method carried out for the surgery system 20 of FIG. 1, and FIGS. 3-8 are diagrams illustrating the steps of the method of FIG. 2, according to an embodiment of the present invention. The pre-planning component described by the flow chart is typically implemented prior to performance of the invasive surgery procedure on patient 22, and determines an optimal path to be followed by invasive probe in the procedure. The pre-planning is assumed to be performed by physician 54.

In an initial step 100 of the flow chart, a computerized tomography (CT) X-ray scan of the nasal sinuses of patient 22 is performed, and the data from the scan is acquired by processor 40. As is known in the art, the scan comprises two-dimensional X-ray “slices” of the patient, and the combination of the slices generates three-dimensional voxels, each voxel having a Hounsfield unit, a measure of radiodensity, determined by the CT scan.

In an image generation step 102, physician 54 displays results of the scan on screen 56. The results may be displayed as a series of two-dimensional (2D) slices, typically along planes parallel to the sagittal, coronal, and/or transverse planes of patient 22, although other planes are possible. The orientation of the planes may be selected by the physician.

The displayed results are typically gray scale images; an example provided in FIG. 3 is a slice parallel to the coronal plane of patient 22. The values of the gray scales, from black to white, may be correlated with the Hounsfield unit (HU) of the corresponding voxels, so that, as applies to the image of FIG. 3, air (HU=−1000) may be assigned to black, and dense bone (HU=3000) may be assigned to white.

Apart from the values for air and water, which by definition are respectively −1000 and 0, the value of the Hounsfield unit of any other substance or species, such as dense bone, is dependent, inter alia, on the spectrum of the irradiating X-rays used to produce the CT scans referred to herein. In turn, the X-ray spectrum depends on a number of factors, including the potential in kV applied to the X-ray generator, as well as the composition of the anode of the generator. For clarity in the present disclosure, the values of Hounsfield units for a particular substance or species are assumed to be as given in Table I below.

TABLE I

| Substance/Species | Hounsfield Unit |
|---|---|
| Air | −1000 |
| Soft tissue | −300 to −100 |
| Fat | −50 |
| Water | 0 |
| Blood | +30 to +45 |
| Dense bone | +3000 |

The HU numerical value for a particular species (other than air and water) as given in Table I are to be understood as being purely illustrative, and those having ordinary skill in the art will be able to modify these illustrative values without undue experimentation, according to the species and the X-ray machine used to generate the CT images referred to herein.

Typically, a translation between HU values and gray scale values is encoded into a DICOM (Digital Imaging and Communications in Medicine) file of the CT scan output from a given CT machine. For clarity in the following description, the correlation of HU=−1000 to black, and HU=3000 to white, and correlations of intermediate HU values to corresponding intermediate gray levels is used, but it will be understood that this correlation is purely arbitrary. Those having ordinary skill in the art will be able to adapt the description herein to accommodate other correlations between Hounsfield units and gray levels, and all such correlations are assumed to be comprised within the scope of the present invention.

In a marking step 104 the physician marks an intended start point to insert probe 28 into the patient, and an intended target point, where the distal end of the probe is to terminate. The two points may be on the same 2D slice, or alternatively, on different slices. Typically, both points are in air, i.e., where HU=−1000, and the termination point is usually, but not necessarily, at a junction of air with liquid or tissue shown in the slice. (An example where the termination point is not at such a junction is when the point is in the middle of an air-filled chamber.) FIG. 4 illustrates a start point 150 and a termination point 152 that are marked on the same 2D slice by the physician, and for clarity these points are assumed, except where otherwise stated, to be the points used in the remaining description of the flow chart. Typically the start and termination points are displayed in a non-gray scale color, for example, red.

In a permissible backward path definition step 106, the physician defines ranges of Hounsfield units which the backward path-finding algorithm, referred to below, uses as acceptable voxel values to find a backward path from termination point 152 to start point 150. The defined range typically includes HUs equal to −1000, corresponding to air or a void in the path; the defined range may also include HUs greater than −1000, for example, the range may be defined as given by expression (1):

{HU|−1000≤HU≤U} (1)

where U is a value selected by the physician.

For example, U may be set to +45, so that the path taken may include water, fat, blood, and soft tissue, as well as air or a void.

There is no requirement that the defined range of values be a continuous range, and the range may be disjoint, including one or more sub-ranges. In some embodiments a sub-range may be chosen to include a specific type of material. An example of a disjoint range is given by expression (2):

{HU|HU=−1000 or A≤HU≤B} (2)

where A, B are values selected by the physician.

For example, A, B may be set to be equal to −300 and −100 respectively, so that the path taken may include air or a void and soft tissue.

The selection method for the range of HUs may be by any convenient method known in the art, including, but not limited to, number, and/or name of material, and/or gray scale. For example, in the case of selection by gray scale, physician 54 may select one or more regions of the CT image, and the HU equivalents of the gray scale values in those selected regions are included in the acceptable range of HUs for voxels of the backward path to be determined by the path-finding algorithm.

In the case of selection by name, a table of named species may be displayed to the physician. The displayed table is typically similar to Table I, but without the column giving Hounsfield unit values. The physician may select one or more named species from the table, in which case the HU equivalents of the selected named species are included in the acceptable range of HUs for voxels of the path to be determined by the path-finding algorithm.

In a path finding step 108, processor 40 implements a path-finding algorithm to find one or more shortest backward paths between termination point 152 and start point 150 to be followed by probe 28. The algorithm assumes that traversable voxels in the path include any voxels in the HU range defined in step 106, and that voxels having HU values outside this defined range act as barriers in any found path. While the path-finding algorithm used may be any algorithm that is able to determine a shortest path within a three-dimensional maze, the inventors have found that the flood fill algorithm, Dijkstra's algorithm, or an extension such as the A* algorithm, give better results in terms of accuracy and speed of computation in determining the shortest backward path, as opposed to other algorithms such as the Floyd-Warshall algorithm or variations thereof.

In some embodiments, the path-finding step includes accounting for the mechanical properties and dimensions of probe 28. For example, in a disclosed embodiment, probe 28 may be limited, when it bends, to a range of possible radii of curvature. In determining possible paths to be followed by the probe, the processor ensures that no portion of the path defines a radius less than this range of radii.

In a further disclosed embodiment, the processor includes accounting for probe mechanical properties that permit different ranges of radii of curvature for different portions of the probe. For example, the end of a possible path may have a smaller radius of curvature than the possible radii of curvature of a proximal part of the probe. However, the distal end of the probe may be more flexible than the proximal part, and may be flexible enough to accommodate the smaller radius of curvature, so that the possible path is acceptable.

In considering the possible radii of curvature of the probe, and the different radii of curvature of possible paths, the processor takes into account which portions of a path need to be traversed by different portions of the probe, as well as the radii of curvature achievable by the probe, as the distal end of the probe moves from start point 150 to termination point 152.

In a yet further disclosed embodiment, the processor ensures that a path diameter D is always larger than a measured diameter d of probe 28. The confirmation may be at least partially implemented, for example, by the processor using erosion/dilation algorithms, as are known in the art, to find voxels within the ranges defined in step 106.

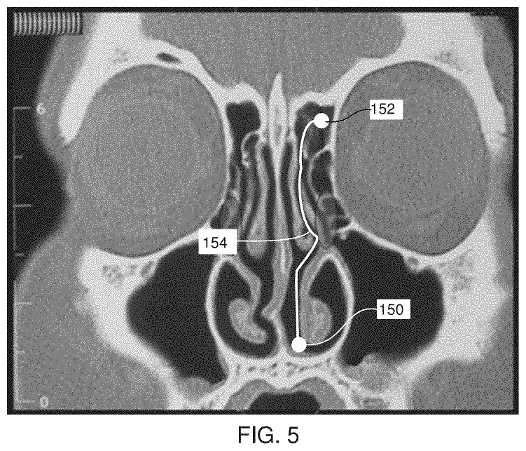

In an overlay step 110, the shortest backward path found in step 108 is overlaid on an image that is displayed on screen 56. FIG. 5 illustrates a shortest backward path 154 between termination point 152 and start point 150 that has been overlaid on the image of FIG. 4. Typically, path 154 is displayed in a non-gray scale color, which may or may not be the same color as the termination and start points. In case step 108 finds more than one shortest backward path, all such paths may be overlaid on the image, typically in different non-gray scale colors.

Typically, the found backward path traverses more than one 2D slice, in which case the overlay may be implemented by incorporating the found path into all relevant 2D slices, i.e., slices through which the path traverses. Alternatively or additionally, an at least partially transparent 3D image may be generated from the 2D slices of the scan, and the backward found path may be overlaid on the 3D image. The at least partially transparent 3D image may be formed on a representation of an external surface of patient 22, as is described in more detail below.

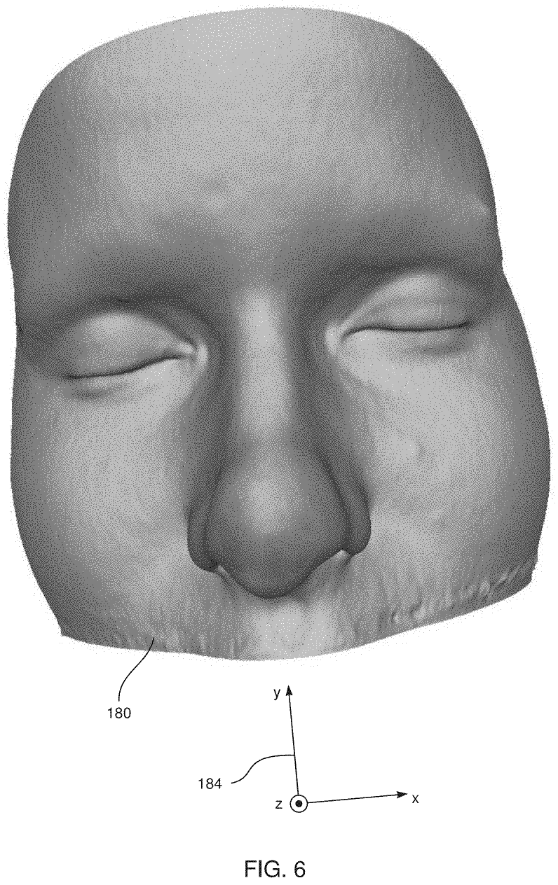

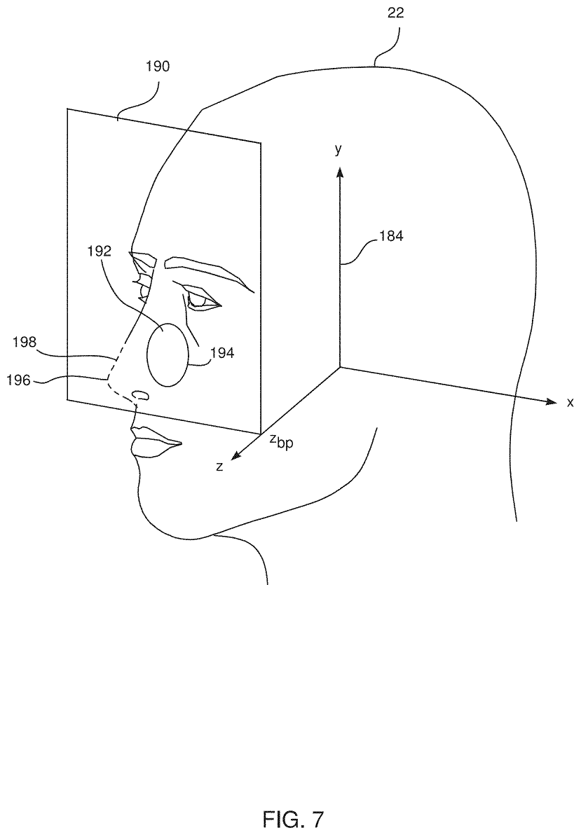

FIG. 6 is a representation of an external surface 180 of patient 22, according to an embodiment of the present invention. Processor 40 uses the CT scan data acquired in step 100 to generate the representation of the external surface, based on air having an HU value of −1000 and skin having a significantly different HU value. By way of example, representation 180 is assumed to be formed on a plane parallel to the coronal plane of the patient, i.e., parallel to an xy plane of a frame of reference 184 defined by the patient, the axes of which are also drawn in FIG. 6 and in FIG. 7 below.

FIG. 7 schematically illustrates a boundary plane 190 and a bounding region 192, according to an embodiment of the present invention. Under directions from physician 54, processor 40 delineates regions of representation 180 which are to be rendered transparent, and those which are to be left “as is.” In order to perform the delineation, the physician defines both boundary plane 190 and bounding region 192 in the boundary plane, using a bounding perimeter 194 for the region.

For clarity, the following description assumes that the boundary plane is parallel to an xy plane of frame of reference 184, as is illustrated schematically in FIG. 7, and that it has an equation given by:

z=z_{bp} (3)

As described below, processor 40 uses the boundary plane and the bounding region to determine which elements of surface 180 are to be rendered locally transparent, and which elements are not to be so rendered.

Processor 40 determines elements of surface 180 (FIG. 6) having values of z≥z_{bp}, and that, when projected along the z-axis, lie within area 192. The processor then renders the elements transparent so that, consequently, these elements are no longer visible in surface 180. For example, in FIG. 7 a tip 196 of the nose of patient 22 has a value z≥z_{bp}, so a broken line 198 in the vicinity of the patient's nose tip illustrates parts of external surface 180 that are no longer visible when the image of the surface is presented on screen 56.

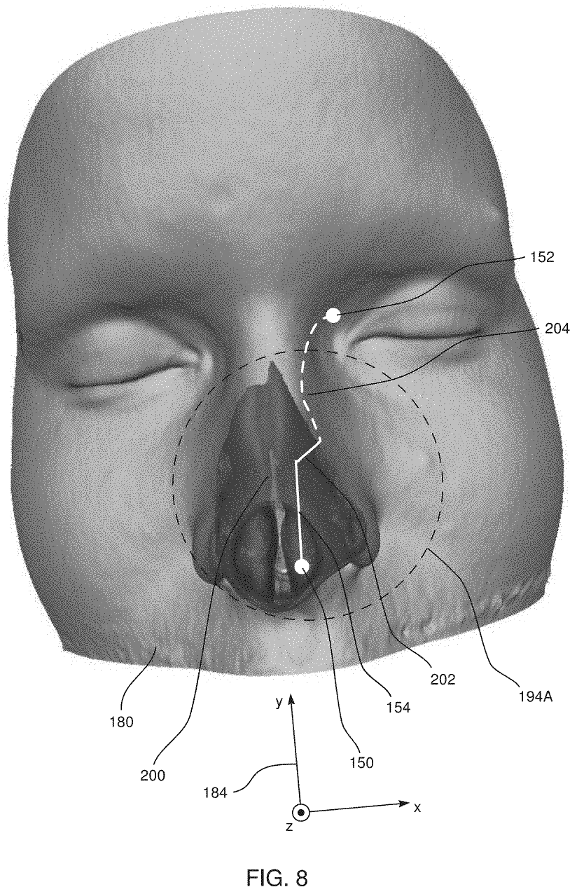

In consequence of the above-defined elements being rendered transparent, elements of surface 180, having values of z<z_{bp} and that when projected along the z-axis lie within area 192 are now visible, so are displayed in the image. Prior to the local transparent rendering, the “now visible” elements were not visible since they were obscured by surface elements. The now visible elements include elements of shortest backward path 154, as is illustrated in FIG. 8.

FIG. 8 schematically illustrates surface 180 as displayed on screen 56 after the local transparency rendering of the elements of the surface within area 170. For clarity, a broken circle 194A, corresponding to perimeter 194 (FIG. 6), has been overlaid on the image, and frame of reference 184 is also drawn in the figure. Because of the transparent rendering of elements within circle 194A, an area 200 within the circle now shows internal structure, derived from the CT tomographic data received in step 100, of subject 22.

Shortest backward path 154 has also been drawn in FIG. 8. Because of the transparent rendering of elements within circle 194A, a portion of the path is now visible in the image of surface 180, and has been drawn as a solid white line 202. The portion of the path that is invisible, because it is hidden by elements of surface 180 that have not been rendered transparent, is shown as broken white line 204.

It will be appreciated that in the case illustrated in FIGS. 6 and 8 screen 56 is in an xy plane, so that the screen acts as a “virtual camera” of a viewer looking towards surface 180 along a z axis.

The description above provides one example of the application of local transparency to viewing a shortest backward path derived from tomographic data, the local transparency in this case being formed relative to a plane parallel to the coronal plane of the subject. It will be understood that because of the three-dimensional nature of the tomographic data, it may be manipulated so that embodiments of the present invention may view the shortest backward path using local transparency formed relative to substantially any plane through patient 22, and that may be defined in frame of reference 184.

In forming the local transparency, the dimensions and position of the bounding plane and the bounding region may be varied to enable the physician to view the shortest backward path, as well as internal structures in the vicinity of the path.

The physician may vary the direction of the bounding plane, for example to enhance the visibility of particular internal structures. While the bounding plane is typically parallel to the plane of the image presented on screen 56, this is not a requirement, so that if, for example, the physician wants to see more detail of a particular structure, the bounding plane may be rotated so that it is no longer parallel to the image plane.

In some cases the range of HU values/gray scales selected in step 106 includes regions other than air, for example, regions that correspond to soft tissue and/or mucous. The backward path found in step 108 may include such regions, and in this case, for probe 28 to follow the path, these regions may have to be cleared, for example by debriding. In an optional warning step 112, the physician is advised of the existence of regions within backward path 154 that are not in air, for example by highlighting a relevant section of the path, and/or by other visual or auditory cues.

While the description above has assumed that tomography is acquired by CT, or X-ray scans, it will be understood that embodiments of the present invention comprise finding a shortest backward path using magnetic resonance imaging (MRI) tomography images as well.

Thus, referring back to step 106 of the flow chart, because Hounsfield units are not applicable to MRI images, the physician instead defines ranges of gray scale values (of the MRI intensities in the MRI images) which the path-finding algorithm uses as acceptable voxel values in finding a path from the start point to the termination point. In step 108, the path-finding algorithm assumes that traversable voxels in the path include any voxels having gray scales in the range defined in step 106, and that voxels having gray scale values outside this defined range act as barriers in any path found. Other changes to the description above to accommodate using MRI images rather than X-ray CT images will be apparent to those having ordinary skill in the art, and all such changes are to be considered as comprised within the scope of the present invention.

Although the embodiments described herein mainly address otolaryngology applications, the methods and systems described herein can also be used in other applications, such as in minimally invasive probing of target locations in organs of the abdomen and thorax.

It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method, comprising:
receiving a medical imaging scan of at least a part of a body of a patient;
identifying voxels of the scan that correspond to regions in the body that are traversable by a probe inserted therein;
displaying the scan on a screen and marking thereon selected termination and start points for the probe;
using a processor, finding a backward path from the termination point to the start point comprising a connected set of the identified voxels; and
visualizing the backward path on the screen in association with the scan.

2. The method according to claim 1, wherein visualizing the backward path comprises:
using the scan to generate a representation of an external surface of the body and displaying the representation on the screen; and
rendering an area of the external surface surrounding the path locally transparent in the displayed representation, so as to make visible on the screen an internal structure of the body in a vicinity of the backward path.

3. The method according to claim 1, wherein identifying the voxels of the scan comprises selecting mucous as a traversable species.

4. The method according to claim 1, wherein identifying the voxels of the scan comprises identifying soft tissue as a traversable species.

5. The method according to claim 1, wherein the medical imaging scan is a computerized tomography (CT) scan, and wherein identifying the voxels of the scan comprises defining a range of Hounsfield units for voxels.

6. The method according to claim 1, wherein the medical imaging scan is a magnetic resonance (MR) scan, and wherein identifying the voxels of the scan comprises defining a range of MR image intensities for voxels.

7. The method according to claim 1, wherein finding the backward path comprises ensuring that no portion of the path comprises a radius of curvature smaller than a range of possible radii of curvature of the probe.

8. The method according to claim 1, wherein finding the backward path comprises ensuring that a path diameter is always larger than a diameter of the probe.

9. The method according to claim 1, wherein finding the backward path comprises finding a shortest path from the termination point to the start point.

10. The method according to claim 9, wherein finding the shortest backward path comprises using Dijkstra's algorithm or an extension thereof.

11. The method according to claim 1, wherein finding the backward path comprises ensuring that the probe is not required to traverse a portion of the path having a path radius curvature smaller than a probe radius of curvature achievable at the portion.

12. Apparatus, comprising:
a screen configured to display a medical imaging scan of at least a part of a body of a patient; and
a processor configured to:
receive the scan;
identify voxels of the scan that correspond to regions in the body that are traversable by a probe inserted therein;
mark on the screen selected termination and start points for the probe;
find a backward path from the termination point to the start point comprising a connected set of the identified voxels; and
visualize the backward path on the screen in association with the scan.

13. The apparatus according to claim 12, wherein the processor is configured to:
use the scan to generate a representation of an external surface of the body and display the representation on the screen; and
render an area of the external surface surrounding the path locally transparent in the displayed representation, so as to make visible on the screen an internal structure of the body in a vicinity of the path.

14. The apparatus according to claim 12, wherein the processor is configured to identify the voxels of the scan by selecting mucous as a traversable species.

15. The apparatus according to claim 12, wherein the processor is configured to identify the voxels of the scan by identifying soft tissue as a traversable species.

16. The apparatus according to claim 12, wherein the medical imaging scan is a computerized tomography (CT) scan, and wherein the processor is configured to identify the voxels of the scan by defining a range of Hounsfield units for voxels.

17. The apparatus according to claim 12, wherein the medical imaging scan is a magnetic resonance (MR) scan, and wherein the processor is configured to identify the voxels of the scan by defining a range of MR image intensities for voxels.

18. The apparatus according to claim 12, wherein the processor is configured to find the backward path by ensuring that no portion of the path comprises a radius of curvature smaller than a range of possible radii of curvature of the probe.

19. The apparatus according to claim 12, wherein the processor is configured to find the backward path by ensuring that a path diameter is always larger than a diameter of the probe.

20. The apparatus according to claim 12, wherein the processor is configured to find the backward path by finding a shortest path from the start point to the termination point.

21. The apparatus according to claim 20, wherein the processor is configured to find the shortest backward path by using Dijkstra's algorithm or an extension thereof.

22. The apparatus according to claim 12, wherein the processor is configured to find the backward path by ensuring that the probe is not required to traverse a portion of the path having a path radius curvature smaller than a probe radius of curvature achievable at the portion.
